(19) United States
(12) Plant Patent Application Publication
(10) Pub. No.: US 2003/0213035 P1
(43) Pub. Date: Nov. 13, 2003

(54) Black walnut tree named 'Beineke 6'

(76) Inventor: Walter Beineke, West Lafayette, IN (US)

Correspondence Address: BARNES & THORNBURG, P.O. BOX 2786, CHICAGO, IL, 60690-2786, US

(21) Appl. No.: 10/141,094

(22) Filed: May 8, 2002

(51) Int. Cl. A01H 5/00

(52) U.S. Cl. PLT/154

(57) ABSTRACT

A new and distinct cultivar of black walnut tree (Juglans nigra L.) which is distinctly characterized by extremely rapid growth rate, very strong central stem tendency, and excellent straightness, thereby producing excellent timber qualities. The new variety has good nut bearing qualities. Nut crops are abundant and annual. This new variety of black walnut tree (Juglans nigra L.) was discovered by the applicant near South Raub, Tippecanoe County, Ind. in a black walnut planting of seedling progeny from previously selected trees for outstanding timber producing potential. This selection has been designated as BW502, a seedling progeny of patented Purdue-1 (U.S. Plant Pat. No. 4543) in records maintained by the applicant on the performance of the selection and grafts made from the selection and will be known henceforth as ‘Beineke 6.’

### BACKGROUND OF THE INVENTION

This new variety of black walnut tree (Juglans nigra L.) was discovered by the applicant near South Raub, Tippecanoe County, Ind. in a black walnut planting of seedling progeny from previously selected trees for outstanding timber producing potential. This selection has been designated as BW502, a seedling progeny of patented Purdue-1 (U.S. Plant Pat. No. 4543) in records maintained by the applicant on the performance of the selection and grafts made from the selection and will be known henceforth as ‘Beineke 6.’

### SUMMARY OF THE INVENTION

A new and distinct cultivar of black walnut tree (Juglans nigra L.) which is distinctly characterized by extremely rapid growth rate, very strong central stem tendency, and excellent straightness, thereby producing excellent timber qualities. The new variety has good nut bearing qualities. Nut crops are abundant and annual.

After the original clone was selected, and assigned an identity number of BW502 the aforesaid tree was reproduced by collecting scions from it and grafting these onto common black walnut rootstocks at American Forestry Technology Company, West Lafayette, Ind. These asexual reproductions ran true to the parent tree and to each other in all respects.

### DESCRIPTION OF THE DRAWINGS

FIG. 1 is a photograph showing the timber form of ‘Beineke 6.’

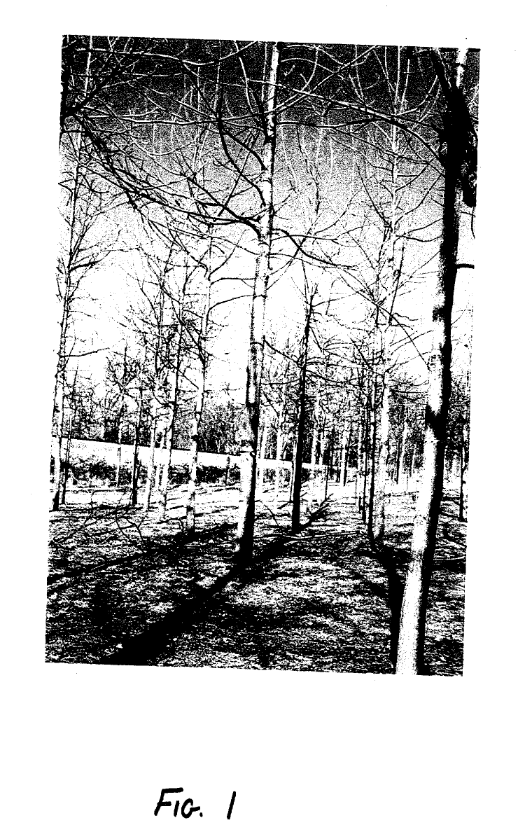

FIG. 2 is a photograph showing the leaves of ‘Beineke 6.’

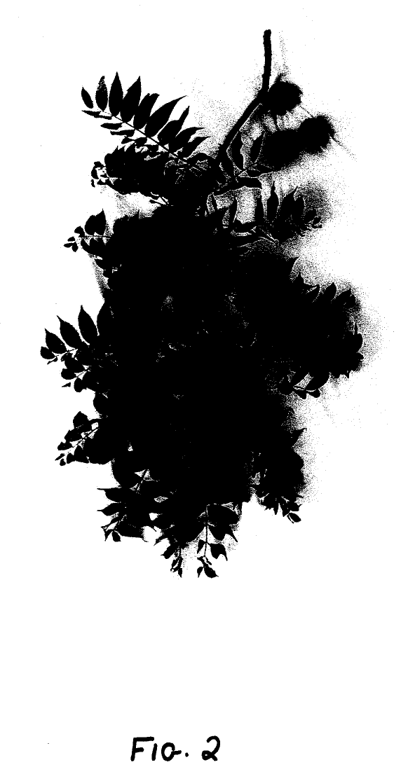

FIG. 3 is a photograph showing the nuts of ‘Beineke 6.’

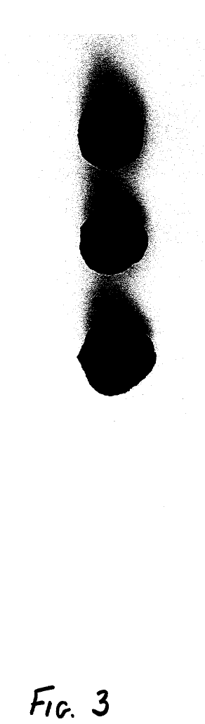

### BOTANICAL DESCRIPTION OF THE PLANTS

The botanical details of this new and distinct variety of walnut tree are as follows:

Tree:

Size.—Large.

Vigor.—Vigorous.

Growth rate.—Very rapid, 38% larger in diameter than the average of Purdue 1 grafts, planted the same year on the same land. Diameter growth rate (at 4½ feet above the ground) averages 0.789 inches per year over 9 years.

Form.—Excellent timber form, as good as Purdue 1 — 51% straighter than average of the entire planting on a rating scale of 1 (excellent) to 5 (very poor), no crooks, very strong central stem tendency — averages 1 on the 1 to 5 scale.

Leaves:

Compound leaves.—Size — Shorter than average; average length — 12.95″.

Leaflets.—Size — Average; average length — 4.05″; average width — 1.33″; average number of leaflets — 20.0— lanceolate; acutely pointed.

Thickness.—thin; Texture — smooth; Margin — serrated; Petiole — short; Color — Topside — dark green; Underside — light green.

Anthracnose resistance.—Fair.

Nut:

Size.—Large; average length — 1.37″; average diameter in suture plane — 1.17″; average diameter cheek to cheek — 1.47″.

Uniformity of size.—Not much variation.

Form.—Rounded; flattened in suture plane.

Blossom end.—Rounded.

Basal end.—Rounded.

Thickness of shell.—Very thick.

Ridges.—Rounded off; not sharp.

Flowering habit:

Age at which trees start producing catkins.—Early.

Number of catkins produced.—Abundant.

Age at which tree starts producing pistillate flowers.—Early.

Number of pistillate flowers produced by young trees.—Abundant.

Lateral shoots producing pistillate flowers.—Yes.

Number of pistillate flowers per inflorescence.—3 to 6.

Nut crop:

Bearing.—Annual.

Productivity.—Medium.

Ripening period.—Late.

Evenness of maturity (period between first and last nuts are ready for harvest).—Even.

Quality.—Good.

Distribution of nuts on tree.—Throughout.

DNA “Fingerprint” for Identification of ‘Beineke 6:’

DNA was isolated from the leaves of Beineke 6. For purposes of DNA fingerprinting, nine highly polymorphic loci from a suite of microsatellites developed by Woeste et al. (2002) were chosen. Microsatellites sizes were checked against previously published standards and verified by a second independent analysis. The “fingerprint” is the collection of microsatellite allele sizes at each locus for ‘Beineke 6.’

DNA was isolated from the leaves of 10 black walnut trees obtained from Walter Beineke using CTAB extraction buffer (50 mM TRIS-HCL, pH 8.0, 20 mM EDTA, pH 8.0, 0.7 M NaCl, 0.4 M LiCl, 2% SDS, 2% TAB, nd 1% PVP). After isolation the DNA from each tree was quantified and diluted with nanopure distilled water to a final concentration of 5 ng/μL. The samples were stored in 96-well plates at 20° C.

For purposes of DNA fingerprinting, nie highly polymorphic loci from a suite of microsatellites developed by Woeste et al. (2002) were chosen. Amplification of each locus was performed with an MJ Rsearch Tetrad Thermocycler (Waltham, Mass.) using 10 μL reactions in 96-well plates. The PCR reaction mix contained 2 μL of the aforementioned black walnut DNA, 5 μL Sigma Taq ReadyMix (Sigma Aldrich, St. Louis, Mo.), 0.4 μL of a 20 pmol mixture of forward and reverse fluorescence labeled primer, and 3 μL PCR grade water supplied with the Sigma ReadyMix. PCR amplification was for 30 cycles of 94° C. for 20 sec, 55° C. for 30 sec, and 72° C. for 1 min. All primers were annealed at 55° C. The products were then held at 4° C. until aliquots could be loaded into 6% Long Ranger (polyacrylamide) denaturing gels (BMA, Rockland, Me.). For each individual 0.5 μL PCR product was added to 0.75 μL blue dextran and 0.25 μL of CXR 350 bp Ladder Standard (Promega, Fitchburg Center, Wis.) in a new 96-well 1 late. The samples were denatured for 2 min at 95° C. and loaded onto a CAL96 96-well laminated membrane comb (The Gel Company, San Francisco, Calif.). Electrophoresis was at 3,000 V, 60 mA, 200 Watts, 50° C. for 2 hours using an ABI 377 (Perkin Elmer) with 36 cm plates and 0.2 mm spacers. The resulting data was analyzed using ABI's GeneScan 3.1.2 and Genotyper 2.5 (Perkin Elmer). Microsatellite sizes were checked against previously published standards and verified by a second independent analysis. The “fingerprint” is the collection of microsatellite allele sizes at each locus for each tree.

Primer Sequences

Primer Sequences

| Locus | Forward | Reverse |
|---|---|---|
| WGA2 | GACGACGAAGGTGTACGGAT | GTACGGCTCTCCTTGCAGTC |
| WGA6 | CCATGAAACTTCATGCGTTG | CATCCCAAGCGAAGGTTG |
| WGA24 | TCCCCCTGAAATCTTCTCCT | TTCTCGTGGTGCTTGTTGAG |
| WGA32 | CTCGGTAAGCCACACCAATT | ACGGGCAGTGTATGCATGTA |
| WG33 | TGGTCTGCGAAGACACTGTC | GGTTCGTCGTTTGTTGACCT |
| WGA86 | ATGCCTCATCTCCATTCTGG | TGAGTGGCAATCACAAGGAA |
| WGA89 | ACCCATCTTTCACGTGTGTG | TGCCTAATTAGCAATTTCCA |
| WGA90 | CTTGTAATCGCCCTCTGCTC | TACCTGCAACCCGTTACACA |
| WGA97 | GGAGAGGAAAGGAATCCAAA | TTGAACAAAAGGCCGTTTTC |

Best interpretation of the current data indicates that the probability that any other black walnut tree would have the collection of microsatellite allele sizes listed is less that 1 in 10⁻¹⁷.

Microsatellites Used to Fingerprint Beineke 6:

| WGA2 | | WGA6 | | WGA24 | | WGA32 | | WGA90 | | WGA86 | | WGA97 | | WGA33 | | WGA89 | |
|---|---|---|---|---|---|---|---|---|---|---|---|---|---|---|---|---|---|
| 150 | 150 | 142 | 142 | 230 | 238 | 181 | 191 | 154 | 170 | 210 | 234 | 159 | 171 | 214 | 214 | 197 | 209 |

### DOCUMENTS CITED

Woeste, K., Burns, R., Rhodes, O., and Michler, C. (2002) (In Press) Thirty polymorphic nuclear microsatellite loci from black walnut. Journal of Heredity.

I claim:

1. A new and distinct variety of black walnut tree named ‘Beineke 6’ substantially as described, which has excellent timber quality, is fast growing, has strong central stem tendency, no sweep, no crooks, and abundant annual nut crops early in the life of the tree.
